(19) United States
(12) Patent Application Publication
(10) Pub. No.: US 2020/0243198 A1
(43) Pub. Date: Jul. 30, 2020

(54) METHOD AND APPARATUS FOR ESTIMATING HUMAN MOTION KINETICS INFORMATION

(71) Applicant: KOREA ADVANCED INSTITUTE OF SCIEMCE AND TECHNOLOGY, DAEJEON (KR)

(72) Inventors: Sukyung PARK, Daejeon (KR); Hyerim LIM, Daejeon (KR); Hyunho JEONG, Daejeon (KR)

(21) Appl. No.: 16/715,007

(22) Filed: Dec. 16, 2019

(30) Foreign Application Priority Data
Jan. 24, 2019 (KR) 10-2019-0009496

(51) Int. Cl. G16H 50/50; A61B 5/11; A61B 5/00; G06N 20/00; G06N 7/00

(52) U.S. Cl. CPC G16H 50/50; A61B 5/1122; A61B 2503/10; G06N 20/00; G06N 7/005; A61B 5/4595

(57) ABSTRACT

A method and apparatus for estimating kinematic information of human motion are provided. The method and apparatus comprises, calculating a length change and angle of a leg spring from a trajectory of human motion by using a compliant walking model modeling a human leg as the spring, and estimating kinematic information including at least one of a ground reaction force, a lower limb angle, an ankle joint trajectory and an ankle joint torque by using the length change and the angle. The compliant walking model comprises an off-centered curvy foot modeling a human foot, a lower limb-ankle spring modeling ranging from the CoM to the ankle joint, and an ankle-foot spring modeling ranging from the ankle joint to the curvy foot.

### CROSS-REFERENCE TO RELATED APPLICATION

This application claims priority to and the benefit of Korean Patent Application No. 10-2019-0009496 filed in the Korean Intellectual Property Office on Jan. 24, 2019, the entire contents of which are incorporated herein by reference.

### BACKGROUND

### (a) Field

The present disclosure relates to a method and apparatus for estimating kinematic information of human motion.

### (b) Description of the Related Art

Recently, as human body dynamics has developed, discussions about obtaining precise kinematic information of human motion by elaborately analyzing human motion have been actively made.

Kinematic information of human body may be used in various cases. For example, the kinematic information may be used to prevent injuries of athletes by calculating loads on lower limb joints during human body movement, to correct walking posture of a person having a wrong walking posture, to rehabilitate an injured body, and the like.

In order to estimate kinematic information of human motion, the conventional compliant walking model that models the human body as a center of mass (CoM) and a spring supporting the CoM. Such a compliant walking model requires measurement of various information such as a vertical ground reaction force, a motion trajectory of center of pressure, a point of measurement change, an acceleration, a body angle, and the like. To measure such information, a large number of devices should be attached to the human body.

By the way, the devices for measuring motion information of the human body are expensive. In addition, if a large number of devices are attached to the human body, the motion of the human body may become unnatural and thereby reliability of the measured information is degraded. In addition, the kinematic information of the human motion that can be estimated by the conventional compliant walking model is limited to a ground reaction force and a joint angle. As such, the conventional compliant walking model is limited at providing any further information about multi-segmental lower limbs that generate oscillatory CoM behaviors and their corresponding ground reaction forces.

### SUMMARY

The present disclosure provides a method and apparatus for estimating various kinematic information of human motion by using a curvy foot compliant walking model. Here, the curvy foot compliant walking model models a human body as a CoM, a ankle joint-CoM spring supporting the CoM at an ankle joint, a foot-ankle spring supporting the ankle joint at a foot and a curvy foot.

According to an embodiment, a method of estimating kinematic information of human motion in an apparatus operated by at least one processor is provided. The method for estimating kinematic information comprises generating a default compliant walking model including a leg spring modeling a leg of a human body as a spring and a curvy foot modeling a human foot in a curved shape, receiving a center of mass (CoM) trajectory of a moving user, generating a compliant walking model for the user by calculating variables related with a length change and an angle of the leg spring, which constitute the default compliant walking model, based on the CoM trajectory, and estimating kinematic information including at least one of a ground reaction force, a lower limb joint angle, an ankle joint trajectory, and an ankle joint torque, by using the compliant walking model for the user. Here, one end of the leg spring is connected to the CoM and the other end of the leg spring is connected to a position apart from a center of the curvy foot by a predefined distance, and the leg spring is divided into a lower limb-ankle spring modeling ranging from the CoM to the ankle joint and an ankle-foot spring modeling ranging from the ankle joint to the curvy foot.

When generating the compliant walking model for the user, the length change and the angle may be calculated by using the CoM trajectory, a radius of curvature of the curvy foot, the predefined distance, and a whole length of the leg spring.

Estimating the kinematic information may comprise calculating a constraint force of the ankle joint by using a constant of the leg spring, the length change, the radius of curvature, an offset value, the angle and an initial length of the leg spring, and calculating the ground reaction force by using the constant, the constraint force, the length change, and the angle.

Estimating the kinematic information may further comprise calculating an ankle joint torque and an ankle joint trajectory, by using the ground reaction force, the radius of curvature, the offset value, the initial length of the leg spring, the length change, the angle, a ratio of a length of the lower limb-ankle spring to that of the ankle-foot spring, and a ratio of a stiffness of the lower limb-ankle spring to that of the ankle-foot spring, respectively.

Estimating the kinematic information may further comprise calculating a length and an angle of a lower limb joint from the CoM trajectory and the ground reaction force, respectively. Here, the length and the angle of the lower limb joint may include a length and an angle of a thigh segment ranging from the CoM to a knee, a length and an angle of a shank segment ranging from the knee to the ankle joint, and a length and an angle of a foot segment ranging from the ankle joint to the curvy foot.

The method for estimating kinematic information may further comprises, before calculating the variables, obtaining the CoM trajectory consisting of position data of each point at a predetermined time interval from a point where a movement of a specific part of human body starts to a point where the movement ends, from a human motion image.

The method for estimating kinematic information may further comprises, after estimating the kinematic information, correcting the estimated kinematic information by using a machine learning model previously trained with the estimated kinematic information.

According to another embodiment, an estimation apparatus comprises a memory that stores a default compliant walking model including a leg spring modeling a leg of a human body as a spring and a curvy foot modeling a human foot in a curved shape, and a program for estimating kinematic information from a trajectory of a center of mass (CoM) of the human body, and at least one processor that executes the program. The program includes instructions to receive a trajectory of the CoM of a moving user, to generate a compliant walking model for the user by calculating variables related with a length change and an angle of the leg spring, which constitute the default compliant walking model, based on the CoM trajectory, and to estimate kinematic information including at least one of a ground reaction force, a lower limb joint angle, an ankle joint trajectory, and an ankle joint torque, by using the compliant walking model for the user. Here, one end of the leg spring is connected to the CoM and the other end of the leg spring is connected to a position apart from a center of the curvy foot by a predefined distance, and the leg spring is divided into a lower limb-ankle spring modeling ranging from the CoM to the ankle joint and an ankle-foot spring modeling ranging from the ankle joint to the curvy foot.

The program may include instructions to calculate a length change of the leg spring, and an angle of the leg spring moved from a vertical line connecting the CoM and ground surface during human motion, by using a two-dimensional position data, a radius of curvature of the curvy foot, the predefined distance and a whole length of the leg spring.

According to embodiments, by using a compliant walking model modeling a curvy foot representing a foot connected to a leg of a human body, more various kinematic information of human motion may be estimated than a compliant walking model where the curvy foot is not modeled.

In addition, according to embodiments, by estimating the CoM trajectory from a human motion image based on image analysis techniques, the CoM trajectory may be easily estimated without requiring a large number of measurement devices for measuring human motion as conventionally.

### BRIEF DESCRIPTION OF THE DRAWINGS

FIG. 1 is a block diagram showing a configuration of an apparatus for estimating kinematic information of human motion according to an embodiment of the present disclosure.

FIG. 2 is diagram showing a trajectory of a center of mass (CoM) according to an embodiment of the present disclosure.

FIG. 3 is a graph showing a trajectory of a CoM according to an embodiment of the present disclosure.

FIG. 4 is a diagram for describing a human walking model according to an embodiment of the present disclosure.

FIG. 5 is a diagram for describing parameters of a human walking model according to an embodiment of the present disclosure.

FIG. 6 is a diagram for describing parameters of a human walking model according to another embodiment of the present disclosure.

FIG. 7 is a diagram for describing parameters of a human walking model according to yet another embodiment of the present disclosure.

FIG. 8 is a flowchart showing a method for estimating kinematic information of a human motion according to an embodiment of the present disclosure.

FIG. 9 is a diagram for explaining a correction process of the kinematic information of human motion according to an embodiment of the present disclosure.

FIG. 10 is a block diagram showing hardware configuration of an apparatus for estimating kinematic information of human motion according to an embodiment of the present disclosure.

### DETAILED DESCRIPTION OF THE EMBODIMENTS

Hereinafter, embodiments of the present disclosure will be described in detail with reference to the attached drawings so that the person of ordinary skill in the art may easily implement the present disclosure. The present disclosure may be modified in various ways and is not limited to the embodiment described herein. In the drawings, elements irrelevant to the description of the present disclosure are omitted for clarity of explanation, and like reference numerals designate like elements throughout the specification.

Throughout the specification, when a part is referred to “include” a certain element, it means that it may further include other elements rather than exclude other elements, unless specifically indicates otherwise.

In addition, the term such as “ . . . unit”, “ . . . block”, “ . . . module”, or the like described in the specification mean a unit that processes at least one function or operation, which may be implemented with a hardware, a software or a combination thereof.

Hereinafter, a method and an apparatus for estimating kinematics information of human motion according to embodiments of the present disclosure are described with reference to the accompanying drawings.

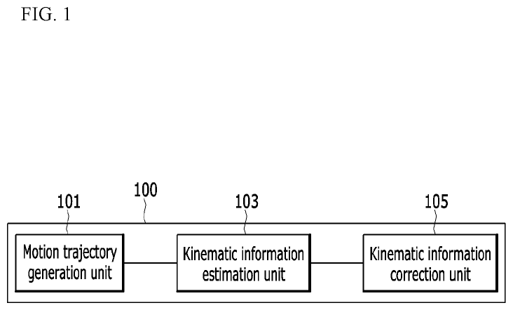

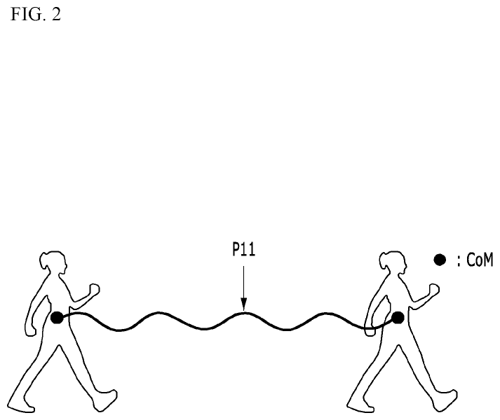

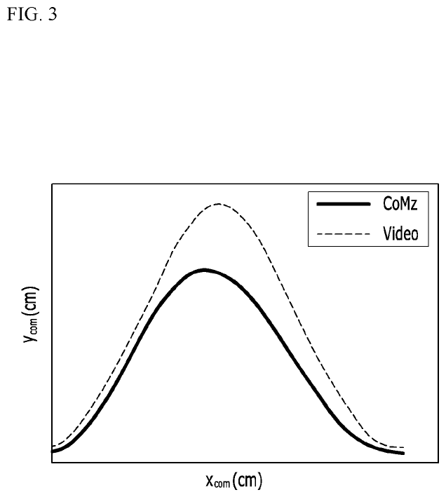

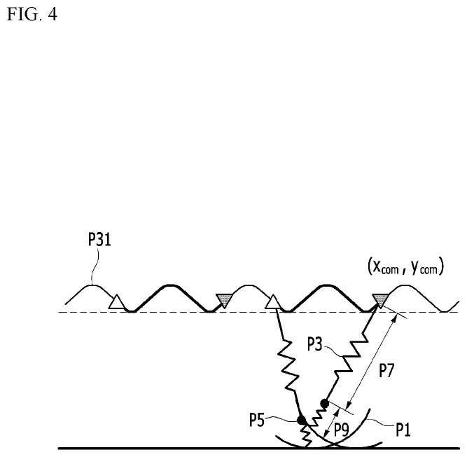

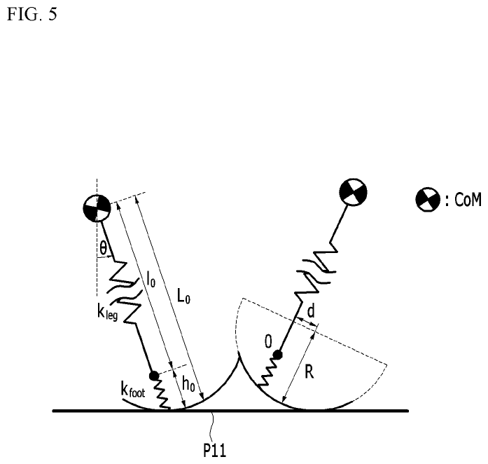

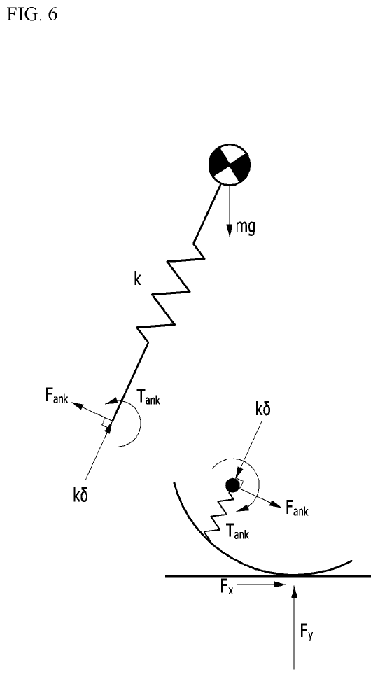

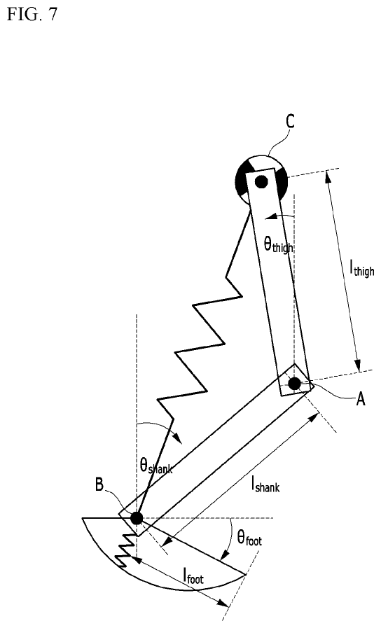

FIG. 1 is a block diagram showing a configuration of an apparatus for estimating kinematic information of human motion according to an embodiment of the present disclosure. FIG. 2 is diagram showing a trajectory of a center of mass (CoM) according to an embodiment of the present disclosure. FIG. 3 is a graph showing a trajectory of a CoM according to an embodiment of the present disclosure. FIG. 4 is a diagram for describing a human walking model according to an embodiment of the present disclosure. FIG. 5 is a diagram for describing parameters of a human walking model according to an embodiment of the present disclosure. FIG. 6 is a diagram for describing parameters of a human walking model according to another embodiment of the present disclosure. And, FIG. 7 is a diagram for describing parameters of a human walking model according to yet another embodiment of the present disclosure.

Referring to FIG. 1, a kinematic information estimation apparatus 100 includes a motion trajectory generation unit 101, a kinematic information estimation unit 103, and a kinematic information correction unit 105.

The motion trajectory generation unit 101 generates a trajectory P11 of a CoM of a human body as shown in FIG. 2.

According to an embodiment, the motion trajectory generation unit 101 generates a motion trajectory of the human body from the human motion image taken by at least one image taking means (not shown). The human motion image may include a walking image, a running image, and the like. The image taking means may be a high speed camera designed for high speed photographing.

The motion trajectory generation unit 101 estimates a trajectory of a feature point of the human body from an RGB image and derives a trajectory of the CoM from the estimated trajectory of the feature point. Here, the feature point of the human body may be selected from body parts having oscillatory motion such as a head, a shoulder, an arm, a waist, a thigh, a knee, an ankle, and the like.

The motion trajectory generation unit 101 specifies a specific body part, for example, a waist, in the RGB motion image of the human body. The motion trajectory generation unit 101 tracks an image area including the waist in the RGB motion image of the human body. Here, for tracking the image area, various image analysis techniques may be used, such as an object area tracking algorithm, a histogram-based mean-shift tracking method, a difference image method where an image fragment of a specific area including a feature in an image is used to identify correlation with another image, a block matching method, and the like. However, the image analysis techniques are not limited thereto. The motion trajectory generation unit 101 extracts a waist position shown in pixel unit from the tracked image area. And, the motion trajectory generation unit 101 may obtain a change length represented by a unit pixel from a height of a subject in the motion image, and may convert the extracted waist position in unit of centimeter using the change length.

At this time, the extracted waist position reflects a movement of not the center of body but the surface of the body. Since the waist position of the body center should be obtained from that of the surface in order to extract the CoM, symmetry of the motion is considered.

In addition, since the CoM reflects mass distribution according to a shape change of a lower limb during movement, the CoM differs from the extracted waist position which is a fixed point on the body. Therefore, the motion trajectory generation unit 101 speculates the shape of the lower limb from the extracted waist position with a stride and a leg length, and derives the CoM in consideration of a mass ratio among each of the body parts. The graph shown in FIG. 3 represents the derived CoM.

The kinematic information estimation unit 103 estimates various kinematic information by using the CoM trajectory output from the motion trajectory generation unit 101 and a compliant working model. The estimated kinematic information includes a ground reaction force (GRF), an ankle joint torque, an ankle joint trajectory, a foot angle, a lower-limb joint angle, and the like.

The kinematic information estimation unit 103 applies a compliant walking model to the CoM trajectory (x_{com}, y_{com}) as shown in FIG. 4. The compliant walking model includes an off-centered curvy foot (hereinafter referred to as “curvy foot”) P1 and a leg spring P3 connecting the curvy foot P1 and the CoM. The leg spring P3 is composed of a lower-limb spring 1 (P7) supporting the CoM at the ankle P5 and a lower-limb spring 2 (P9) supporting the ankle at the curvy foot P1.

Kinematic information estimation unit 103 calculates parameters of the compliant walking model. The parameters of the compliant walking model will be described with reference to FIG. 5 to FIG. 7.

Hereinafter, since the parameters represented by the same symbol in equations are the same, descriptions on the same parameter will not be repeated.

Referring to FIG. 5, L₀ is a length of the unstretched leg spring ranging from the CoM to the ground P11. L₀ satisfying a boundary condition is defined by the following Equation 1.

δ(t⁻)=δ(t⁺)=0 Equation 1

δ is a change length of the leg spring. The kinematic information estimation unit 103 sets initial L₀ as 1 m, and obtains δ from the time point t− of the heel contact to the time point t⁺ of detach. Here, an obtained positive δ at the time of detach means that the initial L₀ is long and an obtained negative δ at the time of detach means that the initial L₀ is short. In consideration of this, the kinematic information estimation unit 103 calculates L₀ that makes the δ at the time of detach be zero, as changing the length of L₀.

L₀ is divided into a length l₀ of the lower limb spring 1 ranging from the CoM to an ankle and a length h₀ of the lower limb spring 2 ranging from the ankle to the curvy foot. The length l₀ of the lower limb spring 1 and the length h₀ of the lower limb spring 2 satisfy the following equation 2.

h₀=r@l₀ Equation 2

Here, r a proportional factor, and h₀ is a fixed value.

The CoM trajectory (x_{com}, y_{com}) is as shown in the following Equation 3.

x_{com}=Rθ+(L₀−\sqrt{R²−d²}−δ)sin θ−d cos θ

y_{com}=R+(L₀−\sqrt{R²−d²}−δ)cos θ+d sin θ Equation 3

Here, state variables are δ and θ. θ is an angle between a leg spring and a line (shown by dotted line in FIG. 5) vertically projected from the CoM, that is, a leg angle. δ is a length change of the leg spring L. ‘R’ is a radius of curvature of the curvy foot, and ‘d’ is an offset amount of the curvy foot. ‘R’ and ‘d’ are fixed values.

The CoM trajectory (x_{com}, y_{com}) is obtained through the motion trajectory generation unit 101. Since L₀ is calculated through Equation 1 and ‘R’ and ‘d’ are fixed values, ‘δ’ and ‘θ’ can be obtained through Equation 3.

FIG. 6 shows degree of freedom of a human body, Fx and Fy represent ground reaction forces (GRFs). The ground reaction force (GRF) is a reaction force applied to a ground contact point of the curvy foot and is headed for the CoM. The GRF is represented by the following equation 4.

$$\{ \begin{matrix}{F}_{x} = k δ sin θ - {F}_{ank} cos θ \\ {F}_{y} = k δ cos θ + {F}_{ank} sin θ\end{matrix} \qquad Equation 4$$

Here, Fₐₙₖ is a constraint force at an ankle joint. Fₐₙₖ is defined by the following Equation 5.

$${F}_{ank} = {ω}_{n}^{2} δ \frac{R sin θ + d}{{L}_{0} - \sqrt{{R}^{2} - {d}^{2}} - δ + R cos θ} \qquad Equation 5$$

Equation 6

Here, ‘G’ is gravity constant. l̅(̅t̅)̅ is an average value of l(t) throughout the stance phase. m is a mass of the leg spring and k is a stiffness of a lower limb joint and is defined by a spring constant. Thus, k is calculated based on body weight.

Tₐₙₖ represents an ankle joint torque and is as shown in the following Equation 7. The Tₐₙₖ is estimated from a constraint torque that aligns ankle-foot segment with the leg throughout the stance phase

$${T}_{ank} = {F}_{x} \cdot \{ R - ( \sqrt{{R}^{2} - {d}^{2}} - \frac{r}{r + 1} {L}_{0} + \frac{1}{ρ + 1} δ ) cos θ + d sin θ \} + {F}_{y} \cdot \{ ( \sqrt{{R}^{2} - {d}^{2}} - \frac{r}{r + 1} {L}_{0} + \frac{1}{ρ + 1} δ ) sin θ + d cos θ \} \qquad Equation 7$$

Here, ‘ρ’ satisfies the following equation 8.

k_{foot}=ρ·k_{leg} Equation 8

Here, k_{leg} represents a stiffness of the lower limb spring 1 (P7 shown in FIG. 4) ranging from the CoM to the ankle, and k_{foot} represents a stiffness of the lower limb spring 2 (P9 shown in FIG. 4) ranging from the ankle to the foot.

In addition, the ankle joint trajectory (xₐₙₖ, yₐₙₖ) is calculated through the following equation 9.

$$\begin{matrix}{x}_{ank} = R θ - ( \sqrt{{R}^{2} - {d}^{2}} - \frac{r}{r + 1} {L}_{0} + \frac{1}{ρ + 1} δ ) sin θ - d cos θ & Equation 9 \\ {y}_{ank} = R - ( \sqrt{{R}^{2} - {d}^{2}} - \frac{r}{r + 1} {L}_{0} + \frac{1}{ρ + 1} δ ) cos θ + d sin θ & \end{matrix}$$

Further, the kinematics of the rest of the lower limb segments such as a foot, a knee and a thigh will be described with reference to FIG. 7.

Referring to FIG. 7, the lower limb segment described in FIG. 4 to FIG. 6 is divided at a knee joint A and an ankle joint B.

l_{thigh} represents a length of a lower limb segment corresponding to the thigh and is a length from the center of mass C to the knee joint A. θ_{thigh} represents a thigh segment angle, which means an angle between the thigh segment and a line (shown as a dotted line) perpendicular to the ground.

The lₛₕₐₙₖ represents a length of a lower limb segment corresponding to a shank, that is, a length from the knee joint A and the ankle joint B. θₛₕₐₙₖ is a shank segment angle, which means an angle between the shank segment and a line (shown as a dotted line) perpendicular to the ground.

l_{foot} is a length from the ankle joint B to a tip of the curvy foot, from the whole lower limb segment. The l_{foot} is calculated as a length from the ankle joint B to the point of contact with the curved surface of the curved foot in the horizontal direction. θ_{foot} is a foot angle and represented by an angle from a horizontal point of the ankle joint B to a point considered as the tip of the foot.

l_{thigh}, θ_{thigh}, lₛₕₐₙₖ, θₛₕₐₙₖ are calculated through the following Equation 10 which uses Equation 3 and Equation 9.

xₐₙₖ−x_{com}=lₛₕₐₙₖ sin θₛₕ+l_{thigh} sin θₜₕ

y_{com}−yₐₙₖ=lₛₕₐₙₖ cos θₛₕ+l_{thigh} cos θₜₕ Equation 10

l_{foot} nd θ_{foot} are calculated through the following Equation 11.

$${θ}_{foot} = θ - tan ( \frac{δ}{( ρ + 1 ) {l}_{foot}} ) \qquad Equation 11$$

As described above, the kinematic information estimation unit 105 may estimate the ankle joint trajectory (xₐₙₖ, yₐₙₖ), the ground reaction force (Fᵣ, F_{y}), the ankle joint torque (Tₐₙₖ), the lower limb joint angles θ_{thigh}, θₛₕₐₙₖ, and θ_{foot}, and the like, through the curvy foot compliant walking model. Therefore, more information for the lower limb segment may be estimated, while the previous compliant walking model may estimate only the ground reaction force and the joint angle.

Referring back to FIG. 1, the kinematic information correction unit 105 corrects the kinematic information estimated by the kinematic information estimation unit 103 using a machine learning algorithm. Here, the machine learning algorithm may include a deep learning model and the like.

The kinematic information correction unit 105 may establish a machine learning model by training the kinematic information estimated from various human motion images in advance. Further, the kinematic information estimated by the kinematic information estimation unit 103 may be corrected by using such machine learning model.

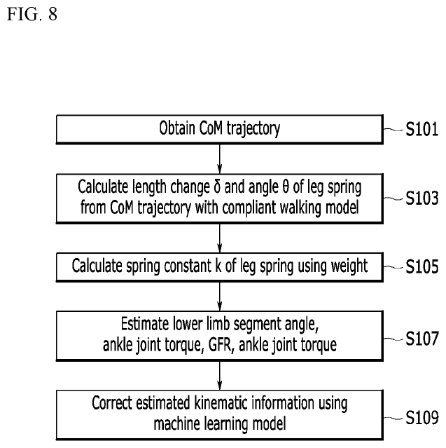

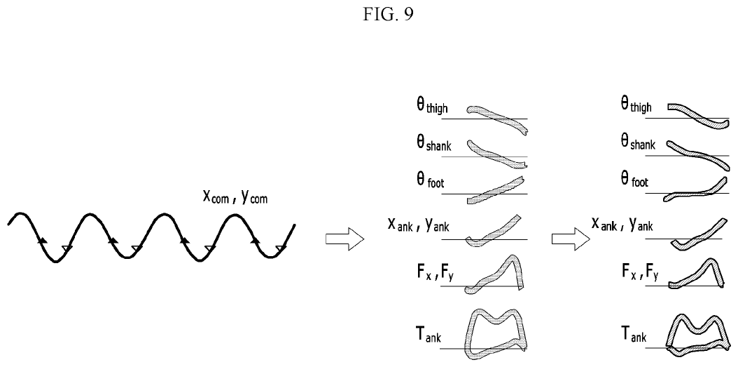

FIG. 8 is a flowchart showing a method for estimating kinematic information of a human motion according to an embodiment of the present disclosure. FIG. 9 is a diagram for explaining a correction process of the kinematic information of human motion according to an embodiment of the present disclosure.

Referring to FIG. 8 and FIG. 9, the motion trajectory generation unit 101 obtains a CoM trajectory (x_{com}, y_{com}), as described above with reference to FIG. 1 to FIG. 7 (S101). The kinematic information estimation unit 103 calculates a change length δ of the leg spring and a leg angle θ from the obtained CoM trajectory, by using the curvy foot compliant walking model (S103).

Here, the curvy foot compliant walking model is divided into a default compliant walking model and a user compliant walking model. Based on the CoM trajectory (x_{com}, y_{com}) received from the motion trajectory generation unit 101, the kinematic information estimation unit 103 establish a user compliant walking model by calculating variables composing the default compliant walking model, that is, δ and θ. Then, by using the user compliant walking model, that is, by applying δ and θ, the kinematic information of a user is estimated. At this time, the kinematic information estimation unit 103 calculates a spring constant k representing stiffness of the leg spring by using weight (S105). Based on the calculated values in steps S103 and S105, the kinematic information estimation unit 103 estimates lower limb joint angles θ_{high}, θₛₕₐₙₖ, and θ_{foot}, an ankle joint trajectory (xₐₙₖ, yₐₙₖ), ground reaction forces (Fₓ, F_{y}), and an ankle joint torque (Tₐₙₖ) (S107).

The kinematic information correction unit 105 corrects the kinematic information estimated by the kinematic information estimation unit 103 using a machine learning model (S109).

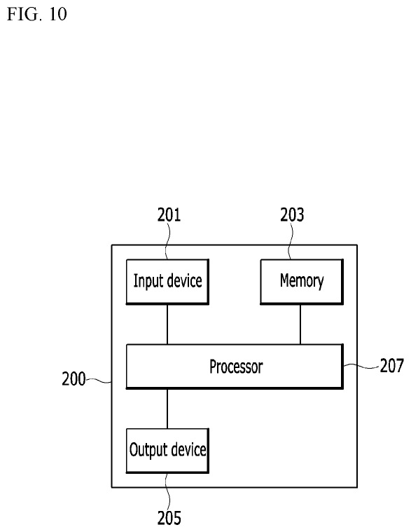

FIG. 10 is a block diagram showing hardware configuration of an apparatus for estimating kinematic information of human motion according to an embodiment of the present disclosure.

Referring to FIG. 10, the apparatus 100 for estimating kinematic information of human motion shown in FIG. 1 may be a computing device 200. The computing device 200 may include an input device 201, a memory 203, an output device 205, and at least one processor 207. The input device 201 and the output device 205 are connected to the at least one processor 207. The input device 201 receives and an image, a sensor signal, and the like and transmits them to the at least one processor 207. The output device 205 outputs estimated and corrected kinematic information of human motion on a display. The memory 203 is connected to the at least one processor 207 and stores a program. The program includes configuration and/or method according to above-described embodiments with reference to FIG. 1 to FIG. 9, that is, instructions to execute operations of the motion trajectory generation unit 101, kinematic information estimation unit 103, and the kinematic information correction unit 105. The program implements the present disclosure in combination with hardware such as the memory 203, the at least one processor 207 and the like.

Embodiments of the present disclosure described above are not implemented only through the apparatus and the method, but may be implemented through a program that realizes functions corresponding to the configuration of the embodiments of the present disclosure or a recording medium on which the program is recorded.

While the present disclosure has been illustrated and descried with reference to embodiments thereof, the right scope of the present disclosure is not limited thereto. Further, it will be understood by a person of ordinary skill in the art that various changes in form and detail may be made therein without departing from the spirit and scope of the present disclosure as defined by the following claims.

What is claimed is:

1. A method of estimating kinematic information of human motion in an apparatus operated by at least one processor, the method comprising:
generating a default compliant walking model including a leg spring modeling a leg of a human body as a spring and a curvy foot modeling a human foot in a curved shape;
receiving a center of mass (CoM) trajectory of a moving user;
generating a compliant walking model for the user by calculating variables related with a length change and an angle of the leg spring, which constitute the default compliant walking model, based on the CoM trajectory; and
estimating kinematic information including at least one of a ground reaction force, a lower limb joint angle, an ankle joint trajectory, and an ankle joint torque, by using the compliant walking model for the user,
wherein one end of the leg spring is connected to the CoM and the other end of the leg spring is connected to a position apart from a center of the curvy foot by a predefined distance, and
wherein the leg spring is divided into a lower limb-ankle spring modeling ranging from the CoM to the ankle joint and an ankle-foot spring modeling ranging from the ankle joint to the curvy foot.

2. The method of claim 1, wherein generating the compliant walking model for the user comprises calculating the length change and the angle by using the CoM trajectory, a radius of curvature of the curvy foot, the predefined distance, and a whole length of the leg spring.

3. The method of claim 2, wherein estimating the kinematic information comprises
calculating a constraint force of the ankle joint by using a constant of the leg spring, the length change, the radius of curvature, an offset value, the angle and an initial length of the leg spring, and
calculating the ground reaction force by using the constant, the constraint force, the length change, and the angle

4. The method of claim 3, wherein estimating the kinematic information further comprises calculating an ankle joint torque and an ankle joint trajectory, by using the ground reaction force, the radius of curvature, the offset value, the initial length of the leg spring, the length change, the angle, a ratio between a length of the lower limb-ankle spring and a length of the ankle-foot spring, and a ratio between a stiffness of the lower limb-ankle spring and a stiffness of the ankle-foot spring.

5. The method of claim 4, wherein estimating the kinematic information further comprises calculating a length and an angle of a lower limb joint from the CoM trajectory and the ground reaction force, respectively, and
wherein the length and the angle of the lower limb joint includes a length and an angle of a thigh segment ranging from the CoM to a knee, a length and an angle of a shank segment ranging from the knee to the ankle joint, and a length and an angle of a foot segment ranging from the ankle joint to the curvy foot.

6. The method of claim 1, further comprising, before calculating the variables, from a human motion image, obtaining the CoM trajectory including position data of each point at a predetermined time interval from a point where a movement of a specific part of human body starts to a point where the movement ends.

7. The method of claim 1, further comprising, after estimating the kinematic information, correcting the estimated kinematic information by using a machine learning model previously trained with the estimated kinematic information.

8. An estimation apparatus comprising:
a memory that stores a default compliant walking model including a leg spring modeling a leg of a human body as a spring and a curvy foot modeling a human foot in a curved shape, and a program for estimating kinematic information from a trajectory of a center of mass (CoM) of the human body; and
at least one processor that executes the program,
wherein the program includes instructions
to receive a trajectory of the CoM of a moving user, and to generate a compliant walking model for the user by calculating variables related with a length change and an angle of the leg spring, which constitute the default compliant walking model, based on the CoM trajectory, and
to estimate kinematic information including at least one of a ground reaction force, a lower limb joint angle, an ankle joint trajectory, and an ankle joint torque, by using the compliant walking model for the user, and
wherein one end of the leg spring is connected to the CoM and the other end of the leg spring is connected to a position apart from a center of the curvy foot by a predefined distance, and
wherein the leg spring is divided into a lower limb-ankle spring modeling ranging from the CoM to the ankle joint and an ankle-foot spring modeling ranging from the ankle joint to the curvy foot.

9. The apparatus of claim 8, wherein the program includes instructions to calculate a length change of the leg spring, and an angle of the leg spring moved from a vertical line connecting the CoM and ground surface during human motion, by using a two-dimensional position data, a radius of curvature of the curvy foot, the predefined distance and a whole length of the leg spring.
